(19) United States
(12) Patent Application Publication
(10) Pub. No.: US 2013/0172283 A1
(43) Pub. Date: Jul. 4, 2013

(54) LYOPHILIZED FORMULATION OF PECTIN-ADRIAMYCIN CONJUGATE AND PREPARATION METHOD THEREOF

(71) Applicants: Xiaohai Tang, Chongqing (CN); Yu Qiu, Chongqing (CN)

(72) Inventors: Xiaohai Tang, Chongqing (CN); Yu Qiu, Chongqing (CN)

(73) Assignee: CHONGQING LUMMY PHARMACEUTICAL CO., LTD., Chongqing (CN)

(21) Appl. No.: 13/695,048

(22) PCT Filed: Apr. 22, 2011
(86) PCT No.: PCT/CN2011/073153

§ 371 Date: Jan. 7, 2013

(30) Foreign Application Priority Data
Apr. 27, 2010 (CN) 201010157856.1
Jun. 3, 2010 (CN) 201010189969.X

(51) Int. Cl. A61K 31/704

(52) U.S. Cl. CPC A61K 31/704
USPC 514/34

(57) ABSTRACT

The invention belongs to the pharmaceutical field, and particularly relates to a lyophilized formulation of pectin-adriamycin conjugate and a preparation method thereof. In order to solve insolubility problem of the pectin-adriamycin conjugate (hereinafter referred to as PAC), improve bioavailability and facilitate preparation, the inventor prepared the PAC into a nanosuspension. However, as long-term stability of the nanosuspension is poor, the inventor proposed to prepare the nanosuspension into a lyophilized formulation, that is, an insoluble pectin-adriamycin conjugate is prepared into a suspension or nanosuspension, and a lyophilized support agent is added to the suspension for lyophilization treatment to prepare the lyophilized formulation. Lyophilized products prepared from the nanosuspension are characterized by enhanced stability of nano-particle size and enhanced stability of drug loading rate, which provides a new solution for clinical application of the PAC.

### FIELD OF THE INVENTION

The invention belongs to the pharmaceutical field, and particularly relates to a lyophilized formulation of pectin-adriamycin conjugate and a preparation method thereof.

### DESCRIPTION OF THE RELATED ART

The Chinese patent application titled “Passive solid tumor-targeted anticancer prodrug and preparation method thereof” with application number 200910311854.0 discloses a passive solid tumor-targeted anticancer prodrug formed by bonding pectin and adriamycin, the preparation method is characterized by reacting small molecular pectin with Mw (Mw is weight-average molecular weight, and one of molecular weight representation methods of high molecular compounds) of 5,000-45,000 with adriamycin to obtain a pectin-adriamycin conjugate with Mw of 100,000-1,000,000, preparing a suspension, and treating the suspension using an ultra-high pressure nano homogenizer to obtain the passive solid tumor-targeted anticancer prodrug with particle size of 100 nm-200 nm and melting point of 220-245°; in which the pectin and the adriamycin are linked by an amido bond, and the pectin and the pectin are linked by an ester bond formed by condensing carboxyl groups and hydroxyl groups of pectin molecules. Specifically, the pectin-adriamycin conjugate is a macromolecular prodrug with thousands of molecular weight coupled by reacting carboxyl groups (—COOH) of pectin molecules with amino groups (—NH₂) of adriamycin to form an amido bond when pectin reacts with adriamycin (ADM) in the presence of dehydrant EDC.HCl, i.e. 1-(3-dimethylaminopropyl)-3-ethylcarbodiimide hydrochloride.

In the patent, the pectin-adriamycin conjugate is called pectin-adriamycin conjugate (hereinafter referred to as PAC), and the inventor found that solubility of the PAC is poor in application of the PAC, and repeated solubility tests showed that the PAC is insoluble. Insoluble drugs have two major problems in clinical application, one problem is low bioavailability due to low solubility of the drugs, and the other problem is that conventional formulation methods have certain restriction on application of the insoluble drugs.

In order to solve insolubility problem of the PAC, the invention provides the following solutions.

### SUMMARY OF THE INVENTION

With regard to insoluble drugs at present, a new formulation form (nanosuspension) has attracted more and more attention. Compared with conventional formulations, the nanosuspension has lots of advantages, for example, water is generally used as a dispersion system in a suspension, and the content of main drugs is high, thus being capable of avoiding adverse reactions resulting from some non-aqueous solvents and making administration by injection possible. According to Ostwald-Freundlich equation (lnC₂/C_{1=2σ}M/RTρ(1/r²⁻¹/r¹), r is particle size, and c is solubility), the particle size of drugs in the suspension is small and specific surface area is large, thus being capable of increasing solubility and bioavailability of the drugs to a certain extent. In addition, if the particle size of drugs is controlled below 200 nm, nano particles can realize targeting in a better manner by virtue of EPR effect. In order to overcome insolubility defect of the PAC, the inventor prepared the PAC into a nanosuspension, and tried to solve the technical problem of insolubility by the technical means so as to achieve the purposes of improving bioavailability and facilitating preparation. Specifically, a method for preparing the insoluble pectin-adriamycin conjugate into a suspension comprises adding 500-900 mg pectin-adriamycin conjugate and a stabilizer to 100 mL sterile water for injection to obtain a mixture, grinding the mixture into a suspension, and then preparing the suspension into a nanosuspension.

However, the inventor found that particles of the prepared nanosuspension easily aggregated due to higher surface free energy, although two stabilizers (e.g. PVP K-30 and poloxamer 188) were added to a formula, long-term stability of the suspension was unsatisfactory. As particle size of nano particles is unstable, the PAC nanosuspension cannot be stored for a long time in a suspension state, thus affecting clinical application effect of the formulation.

In order to overcome instability defect of the PAC nanosuspension, and ensure long-term stability thereof, the inventor proposed to prepare the nanosuspension into a lyophilized formulation. Specifically, an insoluble pectin-adriamycin conjugate is prepared into a suspension or nanosuspension, and a lyophilized support agent is added to the suspension for lyophilization treatment to prepare the lyophilized formulation.

The lyophilized support agent used is at least one of mannitol, dextran, lactose, sucrose, glucose, sorbitol or sodium chloride, and selected based on the principles of loose texture, saturated appearance, good resolubility and no major difference in particle size before and after lyophilization. In order to meet the requirements, the addition amount of the lyophilized support agent is 40-120 mg/mL.

Preferably, 80 mg/mL sucrose is added to the suspension or nanosuspension.

A method of the lyophilization treatment comprises the following steps:

(a) separately filling the suspension in penicillin bottles for prefreezing at −40°-1-80° for 4-8 h; and

(b) placing the penicillin bottles in a lyophilizer for lyophilization.

The preferable prefreezing condition is prefreezing at −40° for 6 h, and the lyophilizer is used for drying for at least 24 h preferably.

The method for preparing the insoluble pectin-adriamycin conjugate into the suspension comprises adding 500-900 mg pectin-adriamycin conjugate and a stabilizer to 100 mL sterile water for injection to obtain a mixture, and grinding the mixture into a suspension. In order to improve bioavailability of the ground suspension, the suspension can be further prepared into a nanosuspension, and preparation of the suspension into the nanosuspension in a high-pressure homogenizer is an effective, conventional, common and convenient treatment mode at present.

The stabilizer used is at least one of PVP, poloxamer, sodium dodecyl sulfate, polysorbate or hydroxypropyl methyl cellulose.

PVP is polyvinylpyrrolidone used as a stabilizer and a hydrophilic adjuvant, and preferably PVP K-30.

Poloxamer (used as a stabilizer and a hydrophilic adjuvant) can be of poloxamer 188 or poloxamer 407, and preferably poloxamer 188.

Sodium dodecyl sulfate (SDS) is an ionic surfactant used as a stabilizer, and can provide charge stabilization effect.

Polysorbate is a nonionic surfactant used as a stabilizer, and preferably polysorbate-80 (Tween 80).

Hydroxypropyl methyl cellulose (HPMC) is a high molecular polymer used as a stabilizer.

Stabilizers for preparing the suspension or nanosuspension can be of the surfactants or the high molecular polymer, can realize stable charge for stabilization.

Specifically, the lyophilized formulation of the invention is prepared by adding 500-900 mg PAC, 3000-6000 mg PVP K-30, 500-900 mg poloxamer 188 and 4000-12000 mg sucrose to 100 mL sterile water for injection before lyophilization. The preferable formula is:

PAC (main drug): 776 mg (drug loading rate is 25.8% converted based on adriamycin equivalent of 2 mg/mL);

PVP K-30 (PVP is polyvinylpyrrolidone used as a stabilizer and hydrophilic adjuvant, K-30 is classification by molecular weight): 4000 mg (w/v, 4%); Poloxamer 188 (188 is model, acting as a stabilizer and hydrophilic adjuvant): 700 mg (w/v, 0.7%);

Sucrose (lyophilized excipient): 80 mg/mL (equivalent to 8000 mg sucrose in each 100 mL);

Sterile water for injection (solvent): 100 mL.

Equivalently, the lyophilized formulation of the invention is prepared by adding 776 mg PAC, 4000 mg PVP K-30, 700 mg poloxamer 188 and 8000 mg sucrose to 100 mL sterile water for injection before lyophilization.

Specifically, the preparation process comprises adding 40-120 mg/mL (preferably 80 mg/mL) lyophilized support agent (sucrose) to the PAC nanosuspension for full ultrasonic dissolution. In order to ensure proper lyophilization of the product, liquid height shall be generally controlled within 2 cm.

A preparation method in the laboratory comprises the following steps: separately filling the PAC nanosuspension to be lyophilized (2 mL/bottle) in 10 mL penicillin bottles, placing the penicillin bottles capped with T shape stoppers (gap shall be reserved to allow moisture to spread out during lyophilization) in a refrigerator at −40°-1-80° (preferably −40°) for prefreezing for 4-8 h (preferably 6 h), and then placing in a lyophilizer for lyophilization for at least 24 h.

The lyophilized product prepared by the method is characterized by reddish orange pie appearance, loose texture, saturated appearance without collapse and good resolubility, complete redissolution after adding formulated amount of sterile water for injection and gently shaking, and substantially no change in property and particle size after redissolution and before lyophilization, with average particle size after redissolution measured with a Malvern laser nano particle size analyzer below 200 nm, and PDI value less than 0.25.

### BRIEF DESCRIPTION OF THE DRAWINGS

FIG. 1 is particle shape of the PAC nanosuspension.

FIG. 2 is particle size of the PAC nanosuspension half a year later.

FIG. 3 is particle size of the PAC nanosuspension. In the suspension, the average particle size of nano particles is 167.3 nm, particle size distribution range is narrow, and PDI value is only 0.141.

### DESCRIPTION OF THE PREFERRED EMBODIMENT

The lyophilized formulation provided in the invention is prepared by preparing an insoluble pectin-adriamycin conjugate into a suspension or nanosuspension, and adding a lyophilized support agent to the suspension for lyophilization treatment.

The method for preparing the insoluble pectin-adriamycin conjugate into the suspension or nanosuspension comprises adding 500-900 mg pectin-adriamycin conjugate and a stabilizer to 100 mL sterile water for injection to obtain a mixture, and grinding the mixture into the suspension.

The suspension can be further prepared into a nanosuspension. Specifically, a high-pressure homogenizer is used for preparing the suspension into the nanosuspension.

The stabilizer used is at least one of PVP, poloxamer, sodium dodecyl sulfate, polysorbate or hydroxypropyl methyl cellulose.

PVP is polyvinylpyrrolidone used as a stabilizer and a hydrophilic adjuvant, and preferably PVP K-30.

Poloxamer (used as a stabilizer and a hydrophilic adjuvant) can be of poloxamer 188 or poloxamer 407, and preferably poloxamer 188.

Sodium dodecyl sulfate (SDS) is an ionic surfactant used as a stabilizer, and can provide charge stabilization effect.

Polysorbate is a nonionic surfactant used as a stabilizer, and preferably polysorbate-80 (Tween 80).

Hydroxypropyl methyl cellulose (HPMC) is a high molecular polymer used as a stabilizer.

Stabilizers for preparing the suspension or nanosuspension can be of the surfactants or the high molecular polymer, and can realize stable charge for stabilization.

Further, the method for preparing the insoluble pectin-adriamycin conjugate into the suspension comprises adding 500-900 mg pectin-adriamycin conjugate, 3000-6000 mg

PVP K-30 and 500-900 mg poloxamer 188 to 100 mL sterile water for injection to obtain a mixture, grinding the mixture into the suspension, and preparing the suspension in the nanosuspension.

Specifically, the following mixture ratio is preferable:

PAC (main drug): 776 mg (solvent in the formula is 100 mL sterile water for injection, based on adriamycin equivalent of 2 mg/mL, 200 mg adriamycin is required, drug loading rate of the PAC is 25.8%, 200/25.8%=775.19 mg, and rounding up to 776 mg);

PVP K-30: 4000 mg (w/v, 4%);

Poloxamer 188: 700 mg (w/v, 0.7%);

Sterile water for injection (solvent): 100 mL.

That is, 500-900 mg pectin-adriamycin conjugate, 3000-6000 mg PVP K-30 and 500-900 mg poloxamer 188 are added to 100 mL sterile water for injection to obtain a mixture, and the mixture is ground into the suspension, and the suspension is prepared into the nanosuspension if required.

The nanosuspension of the invention is prepared by referring to the following method based on the formula:

A. preparing PVP K-30 into PVP K-30 solution with sterile water for injection;

B. dry grinding and evenly mixing PAC and poloxamer 188 (the purpose of grinding is to obtain small and even particle size of the ground PAC), and adding a little PVP K-30 solution for full grinding (the purpose of full grinding is to obtain smaller and evener particle size of the PAC suspension so as to prevent blocking homogenizing valves of the high-pressure homogenizer, with preliminary particle size of the PAC suspension less than 80 μm);

C. adding remaining PVP K-30 solution in several times for grinding (the purpose of such operation is to clean up PAC attached to a mortar so as to prevent heavy loss of raw materials);

D. performing ultrasonic treatment on the ground suspension for full mixing so as to prepare the suspension; and

E. treating the suspension evenly mixed in the step D in a high-pressure homogenizer (EmulsiFlex C-3 high-pressure homogenizer, Avestin Inc., Canada); controlling pressure of the homogenizer at 4000-8000PSI (the purpose of such operation is to provide the homogenizer with an adaptation process so as to prevent occurrence of blockage, preferably 5000PSI) for cycle treatment of samples for 3-10 min (the purpose of such operation is to provide the homogenizer with an adaptation process so as to prevent occurrence of blockage, preferably 3 min), and then keeping the pressure of the homogenizer at 20000-30000PSI (the upper pressure limit of homogenizers currently available on the market is generally 30000PSI, i.e. 200 Mpa, preferably 25000PSI) for cycle treatment of the samples for 15-30 min (in the range, the longer the treatment time is, the better the PDI homogeneity value of nano particles of the nanosuspension is, and the treatment time is 20 min for sake of cost factor) to obtain the PAC nanosuspension.

It shall be noted that in order to prevent rising temperature of the homogenizer during treatment at high pressure from affecting physicochemical properties of the drugs, temperature of a constant heat exchanger homogenizer used is 20-40° (preferably 25°) in the whole treatment. Adriamycin is photosensitive, thus all operations are performed away from light.

Specifically, the following method can be used for preparation in the laboratory:

A. precisely weighing PVP K-30 according to the formulated amount, and preparing solution with formulated amount of sterile water for injection;

B. precisely weighing and placing PAC and poloxamer 188 in an agate mortar, dry grinding for even mixing, and adding a little PVP K-30 solution for full grinding, with particle size less than 80 nm (tests show that the requirement can be met by the mortar within 20 min);

C. adding remaining PVP K-30 solution in several times for grinding, transferring the suspension to a conical flask, cleaning up suspension attached to the mortar with a little PVP K-30 solution, merging the liquid, and performing ultrasonic treatment for full mixing (5 min is ok generally); and

D. Treating pretreated samples (equivalent to adriamycin equivalent of 2 mg/mL) in a high-pressure homogenizer: controlling pressure of the homogenizer at 4000-8000PSI (preferably 5000PSI) for cycle treatment of the samples for 3-10 min (preferably 3 min), then keeping the pressure of the homogenizer at 20000-30000PSI (preferably 25000PSI) for cycle treatment of the samples for 15-30 min (preferably 20 min), and controlling the temperature at 20-40° (preferably 25°).

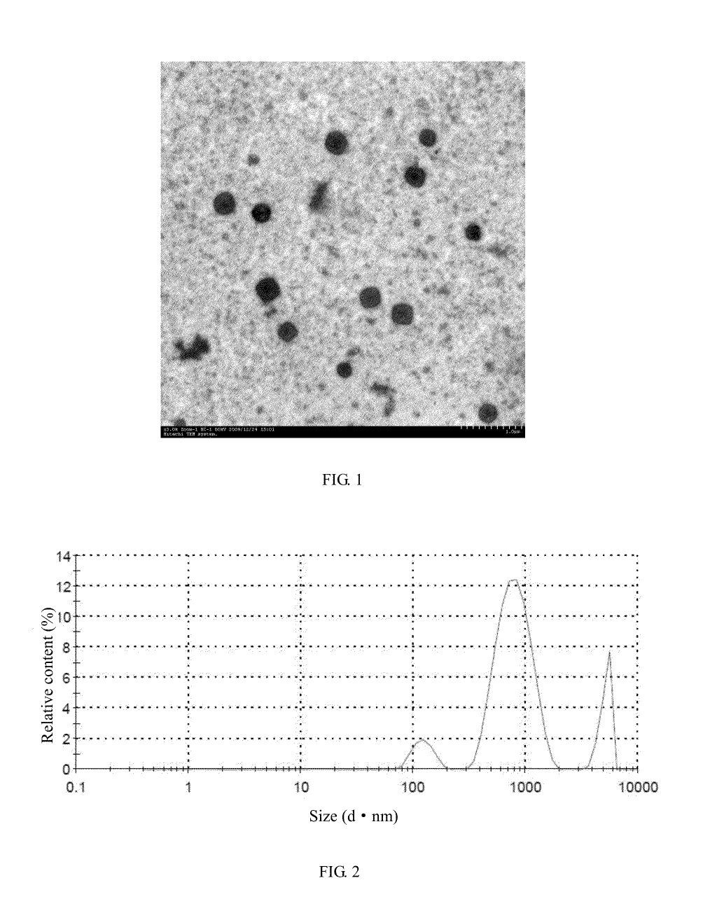

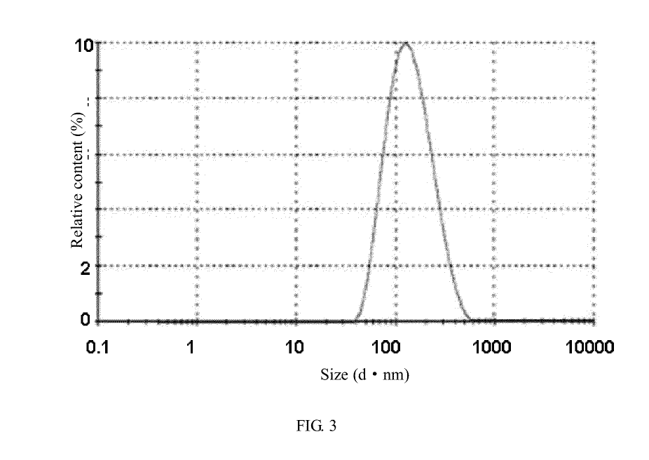

Tests show that the average particle size of the nanosuspension prepared by the method is 167.3 nm, PDI polydispersity value is 0.141 (see FIG. 3), and Zeta potential is −17.5 mV. The result shows that preparation of the PAC nanosuspension by a high pressure homogenization method can meet various quality control indexes, and conform to requirements for particle size of the nanosuspension for injection with smaller particle size. The particle size distribution also meets requirements of the EPR effect for particle size (the particle size is required to be less than 200 nm in the EPR effect), so that the PAC nanosuspension can maximize tumor targeting of the EPR; PDI value is less than 0.2, which indicates that the particle size distribution range is narrow, and proportion of large particles in the suspension can be ensured; and Zeta potential is −17.5 mV, which indicates that the potential stability is higher. Particle shape of the nanosuspension is observed under a transmission electron microscopy (TEM). The TEM can help clearly observe that the particle shape of the PAC nanosuspension is of regular spherical-like shape (see FIG. 1), and particle size is approximate 200 nm, which is substantially consistent with results measured with a laser nano particle size analyzer.

As particles of the nanosuspension easily aggregate due to higher surface free energy, long-term stability of the PAC nanosuspension is determined as follows: separately filling 2 mg/mL freshly prepared PAC nanosuspension in 6 penicillin bottles respectively, keeping away from light, placing the penicillin bottles in a constant temperature & humidity chamber, and controlling the temperature at 25° and humidity at 60%±10%; and sampling a bottle at time intervals of 3 days, 10 days, 25 days, 50 days, 90 days and 6 months respectively to determine particle size and polydispersity. Stability test results of the nanosuspension show that particles of the nanosuspension in a suspension state begin to aggregate and the particle size increases from 167.3 nm to approximate 458 nm after 50 days; and after 6 months, the average particle size increases to 895 nm, particles of the nanosuspension seriously aggregate, nano particles and micron particles coexist (see FIG. 2), serious tailing peak occurs to micron particles in determination of the particle size, and polydispersity becomes very poor (PDI value is 0.602). The phenomenon shows that although large amount of stabilizers is added to the nanosuspension, the long-term stability is still unsatisfactory. Therefore, as the particle size of nano particles is unstable, the PAC nanosuspension cannot be stored for a long time in a suspension state.

The nanosuspension of the invention can be also prepared by the following method:

1. Adding 0.3-0.6 g PAC to 0.4-1.6 g PVP, 2-6 ml glycerol and 40-60 ml 1-3% lecithin solution as a solvent to prepare the suspension, and preparing the suspension into the nanosuspension using a high-pressure homogenizer.

Specifically, 0.468 g macromolecular insoluble pectin-adriamycin conjugate is added with 1 g PVP, 3 ml glycerol and 50 ml 2% lecithin solution as a solvent, and ground to prepare the suspension, and treated in an ultra-high pressure nano homogenizer (T-200D homogenizer manufactured by Hebei Langfang General Machinery Manufacturing Co., Ltd.). The suspension is treated in the ultra-high pressure nano homogenizer for 3 times at 120 mpa for the first time, 180 mpa for the second time and 190 mpa for the third time.

2. Adding 0.3-0.6 g PAC to a mixed solvent of 0.4-1.6 g PVP, 40-60 ml water and DMSO to prepare the suspension, with water:DMSO=0.5-0.85:0.15-0.5, and preparing the suspension into the nanosuspension using the high-pressure homogenizer.

Specifically, 0.468 g macromolecular insoluble pectin-adriamycin conjugate is added with a mixed solvent of 1 g PVP, 50 ml water and DMSO (water:DMSO=0.75:0.25) to prepare the suspension, or further the suspension is prepared into the nanosuspension using the high-pressure homogenizer. Specifically, the high pressure homogenizer is used for treatment to prepare the nanosuspension at pressure not more than 200 mpa each time, (The inventor used T-200D homogenizer manufactured by Hebei Langfang General Machinery Manufacturing Co., Ltd.), specifically, the suspension can be treated in the ultra-high pressure nano homogenizer for 3 times at 120 mpa for the first time, 180 mpa for the second time and 190 mpa for the third time.

The particle size of the nanosuspension prepared by the method is below 200 nm, PDI value is less than 0.25, and absolute potential value is greater than 10 mV. The insoluble drug PAC can be prepared into the nanosuspension by the high pressure homogenization method; and quality test results show that the particle size and polydispersity of nano particles of the nanosuspension meet requirements, and Zeta potential value is large, thus the suspension has higher potential stability, and the particle shape is of regular spherical-like shape observed under the transmission electron microscopy.

Stability main includes physical stability and chemical stability. The physical stability refers to spatial stability of nano particles of the PAC nanosuspension, can be directly characterized by changes of particle size and polydispersity index over time, and can be indirectly illustrated by Zeta potential of a system; and the chemical stability refers to degradation degree of effective drug concentration of the suspension over time. Defect of poor long-term stability of the PAC suspension or nanosuspension thereof is due to poor physical stability of the system.

In order to solve the long-term stability defect of the PAC nanosuspension, the inventor tried the following methods:

(1) Adding large amount of PVP K-30 to increase viscosity of the system, and prevent aggregation of nano particles: PVP K-30 served as a suspending agent to increase the viscosity of the dispersion system. According to stock equation (V=2r²(ρ1−ρ2)g/9n, η-viscosity of a dispersion medium), the larger the viscosity of the system is, the slower the precipitation of the drug particles is, and the better the stability is. The inventor tried to add 8% PVP K-30 in the tests to increase the viscosity of the system, however, with significant increase of the viscosity of the system, pressure on the high-pressure homogenizer during preparation had to be increased, thus aggravating wear of the homogenizer. Even so, the particle size of the finally prepared PAC suspension did not produce good result, the average particle size was larger than 300 nm, and the PDI value was larger than 0.25, thus the method is infeasible.

(2) Adding ionic surfactants such as sodium dodecyl sulfate (SDS) to provide charge stabilization effect, and adding large amount of SDS to the PAC nanosuspension to allow absolute Zeta potential value of the system to be larger than 30Mv. However, the stability test results were not satisfactory, the particle size and the PDI value of the suspension system increased greatly 6 months later, and even obvious precipitation, thus the method is also infeasible.

The second method can not ensure stability of the formulation after long-term storage, thus the method is inadvisable; in the first method, the addition of large amount of PVP increases production cost, and the particle size and the PDI value of the prepared nanosuspension are not optimal.

Finally, the inventor proposed to prepare the nanosuspension into a lyophilized formulation after comprehensively considering all factors, and finally verified that appearance, resolubility, particle size, etc. of the PAC nanosuspension lyophilized product meet requirements.

Lyophilized products shall be added with lyophilized support agents generally, mannitol, dextran, lactose, sucrose, glucose, sorbitol, sodium chloride and various substances can be used as lyophilized support agents, and using amount of the lyophilized support agents are different and not unified depending on different drugs and formulae, and vary widely from milligram to gram. While the substances can be used as lyophilized support agents, different lyophilized support agents have significant impact on appearance, texture, resolubility, changes in particle size of nanosuspension before and after lyophilization, etc. of lyophilized products, therefore, the inventor selected lyophilized support agents capable of providing lyophilized products with loose texture, saturated appearance, good resolubility and no major difference in particle size before and after lyophilization based on appearance, resolubility and difference in particle size before and after lyophilization as indexes. See Table 1 for detailed appearance, redissolution speed, changes in particle size before and after redissolution, etc.

TABLE 1
Screening of various lyophilized support agents

| | Difference in | | | |
|---|---|---|---|---|
| | particle size before | | | |
| Lyophilized | and after | | | Redissolution |
| support agent | redissolution | | Appearance | speed |
| Mannitol | 75-150 | nm | Loosely | Fast |
| | | | saturated | |
| Dextran | >100 | nm | Loosely | Faster |
| | | | cavernous | |
| Lactose | >100 | nm | Loosely | Faster |
| | | | cavernous | |
| Sucrose | <50 | nm | Loosely | Fast |
| | | | saturated | |
| Glucose | >200 | nm | Shrinking | Slow |
| Sorbitol | <50 | nm | Loosely | Faster |
| | | | cavernous | |
| Sodium | 75-150 | nm | Loosely | Fast |
| chloride | | | saturated | |

It can be seen from screening tests that among various lyophilized support agents, the sucrose can provide the best lyophilization effect and optimal appearance, redissolution speed, difference in particle size before and after redissolution, etc., thus the sucrose is intended to be selected as the lyophilized support agent for the nanosuspension.

Meanwhile, the inventor investigated preparation of the PAC nanosuspension into the lyophilized formulation in 6 conditions respectively, i.e. adding lactose, sucrose, glucose, sorbitol, mannitol and not adding any support agent, and the addition amount of 5 support agents were respectively determined as 40 mg/mL, 80 mg/mL and 120 mg/mL. Test results show that in the 6 conditions, products added with 80 mg/mL sucrose are the optimal, and have good appearance and resolubility and almost no difference before and after lyophilization. Therefore, 80 mg/mL sucrose is determined as the preferable lyophilized support agent in the formula.

Specifically, the formula of the lyophilized formulation of the invention is as follows: the lyophilized formulation of the invention is prepared by adding 500-900 mg PAC, 3000-6000 mg PVP K-30, 500-900 mg poloxamer 188 and 4000-12000 mg sucrose to 100 mL sterile water for injection before lyophilization, and preferably by adding 776 mg PAC, 4000 mg PVP K-30, 700 mg poloxamer 188 and 8000 mg sucrose to 100 mL sterile water for injection before lyophilization. The specific preparation process comprises the following steps: preparing the PAC nanosuspension by the method, and then adding 40-120 mg/mL (preferably 80 mg/mL) lyophilized support agent (sucrose) to the PAC nanosuspension for full ultrasonic dissolution. In order to ensure proper lyophilization of the product, liquid height shall be generally controlled within 2 cm. During lyophilization, the nanosuspension to be lyophilized is placed in a −40° refrigerator for refreezing for 6 h, and then placed in a lyophilizer (Thermo Modulyo(savanf), Thermo Scientific Corp. USA) for lyophilization for 24 h.

The following suspension can be also prepared into the lyophilized formulation, that is, 0.468 g PAC is added with 1 g PVP, 3 ml glycerol and 50 ml 2% lecithin solution as a solvent, ground to prepare the suspension (the suspension can be prepared by adding 0.3-0.6 gPAC with 0.4-1.6 g PVP, 2-6 ml glycerol and 40-60 ml 1-3% lecithin solution as a solvent), and treated in an ultra-high pressure nano homogenizer (T-200D homogenizer manufactured by Hebei Langfang General Machinery Manufacturing Co., Ltd.). The suspension is treated in the ultra-high pressure nano homogenizer for 3 times at pressure not more than 200 mpa each time (120 mpa for the first time, 180 mpa for the second time and 190 mpa for the third time), and the prepared suspension is added with 40-120 mg/mL lactose, sucrose, glucose, sorbitol and mannitol to prepare the lyophilized formulation according to the lyophilization process, and sucrose is preferably used as the lyophilized support agent, with preferable addition amount of 80 mg/mL.

Also, 0.468 g PAC can be added with a mixed solvent of 1 g PVP, 50 ml water and DMSO (water:DMSO=0.75:0.25) to prepare the suspension (0.3-0.6 g PAC can be added with a mixed solvent of 0.4-1.6 g PVP, 40-60 ml water and DMSO (water:DMSO=0.5-0.85:0.15-0.5) to prepare a grinding agent so as to prepare the suspension), and treated in an ultra-high pressure nano homogenizer (T-200D homogenizer manufactured by Hebei Langfang General Machinery Manufacturing Co., Ltd.). The suspension is treated in the ultra-high pressure nano homogenizer for 3 times at 120 mpa for the first time, 180 mpa for the second time and 190 mpa for the third time, and the prepared suspension is added with 40-120 mg/mL lactose, sucrose, glucose, sorbitol and mannitol to prepare the lyophilized formulation according to the lyophilization process, and sucrose is preferably used as the lyophilized support agent, with preferable addition amount of 80 mg/mL.

In order to investigate long-term stability of lyophilized powder injection, the following test was performed: sampling 6 bottles of the lyophilized nanosuspension (the lyophilized suspension was prepared based on a preferable lyophilized formulation formula: that is, adding 776 mg PAC, 4000 mg PVP K-30, 700 mg poloxamer 188 and 8000 mg sucrose to 100 mL sterile water for injection), keeping away from light, placing the bottles in a constant temperature & humidity chamber, and controlling the temperature at 25° and humidity at 60%±10%, and sampling a bottle at time intervals of 3 days, 10 days, 25 days, 50 days, 90 days and 6 months respectively to determine particle size and polydispersity. In the same test conditions, the particle size of the PAC nanosuspension lyophilized product was 182.3 nm determined after storage for half a year, and compared with 172.6 nm of freshly prepared suspension lyophilized product, the particle size almost has no major change. The test results show that the stability of the lyophilized product prepared from the suspension is greatly improved compared with that in a suspension storage state. Based on stability of the particle size of the nanosuspension, preparation of the PAC nanosuspension into the lyophilized product can ensure long-term storage stability.

Proposed administration route of the PAC nanosuspension of the invention is intravenous injection, thus the particle size shall be controlled strictly. The 6-month stability test results show that the average initial particle size of the PAC nanosuspension is 167.3 nm, and the average particle size is 895 nm 6 months later; particles seriously aggregate, and nano particles and micron particles coexist. The average initial particle size of the lyophilized PAC nanosuspension is 172.6 nm, the average particle size is 182.3 nm 6 months later, and the particle size almost has no major change. The result shows that the PAC nanosuspension cannot be stored for a long time in a suspension state, and the lyophilized product has good long-term stability and can be stored for a long time. The proposed administration route of the PAC nanosuspension is intravenous injection, thus the particle size shall be controlled strictly. Compared common nanosuspensions with the lyophilized nanosuspension, the latter has absolute advantage in ensuring stability of the particle size.

The inventor also monitored the drug loading rate of the PAC formulation in long-term storage using a method as follows:

### 1. METHOD

1.1 Method for Determination of Drug Loading Rate

1.1.1 Preparation of Standard Curve of Adriamycin Hydrochloride

10.7 mg adriamycin hydrochloride was precisely weighed and placed in a 100 mL volumetric flask, diluted with sterile water for injection to volume as stock solution, and underwent ultrasonic treatment for 5 min for even mixing; 0.2 ml, 0.4 ml, 0.8 ml, 1.2 ml, 1.6 ml and 2 ml adriamycin hydrochloride stock solution were pipetted and placed in 5 ml volumetric flasks respectively, diluted with sterile water for injection to volume, and shook up. Using ultraviolet spectrophotometry, absorbance A values of adriamycin solution were determined at the series of concentration gradients at 480 nm, and linear regression was performed by taking absorbance A as a vertical coordinate and mass concentration C (mg·mL-1) of adriamycin as horizontal coordinate to obtain an equation: A=0.0178C+0.0284, R2=0.9994 (n=3).

1.1.2 Determination of Drug Loading Rate of PAC Nanosuspension

1 mL PAC nanosuspension (prepared according to a preferable solution) was precisely pipetted and placed in a 50 mL volumetric flask (concentration of the PAC nanosuspension was 7.76 mg/mL), diluted with sterile water for injection to volume and shook up, and then proper amount was pipetted to determine absorbance at 480 nm so as to obtain adriamycin concentration Cx with the standard curve. Drug loading rate of the PAC=(Cx/7.76)*100%.

1.1.3 Determination of Drug Loading Rate of PAC Lyophilized Formulation

One bottle of PAC lyophilized formulation (prepared according to a preferable solution) was sampled, and redissolved in 2 mL sterile water for injection into a uniform nanosuspension, 1 mL redissolved PAC nanosuspension was precisely pipetted and placed in a 50 mL volumetric flask (concentration of the PAC nanosuspension was 7.76 mg/mL), diluted with sterile water for injection to volume and shook up, and then proper amount was pipetted to determine absorbance at 480 nm so as to obtain adriamycin concentration Cx with the standard curve. Drug loading rate of the PAC=(Cx/7.76)*100%.

1.2 Monitoring of Drug Loading Rate of PAC Nanosuspension

2 mg/mL freshly prepared PAC nanosuspension was filled in 6 penicillin bottles respectively, kept away from light, placed in a constant temperature & humidity chamber, and the temperature was controlled at 25°, and humidity was controlled at 60%±10%; and a bottle was sampled at time intervals of 3 days, 10 days, 25 days, 50 days, 90 days and 6 months respectively to determine the drug loading rate according to 1.1.2.

1.3 Monitoring of Drug Loading Rate of PAC Lyophilized Formulation

Six bottles of lyophilized PAC nanosuspension (see preparation of lyophilized formulation) were sampled, kept away from light, placed in a constant temperature & humidity chamber, and the temperature was controlled at 25°, and humidity was controlled at 60%±10%; and a bottle was sampled at time intervals of 3 days, 10 days, 25 days, 50 days, 90 days and 6 months respectively to determine the drug loading rate according to 1.1.3.

### 2. RESULTS

The drug loading rate of bulk drugs for preparation of the PAC nanosuspension was 25.8%. The drug loading rate of the PAC nanosuspension tended to decreases gradually during placement, and decreased to 20.3% 6 months later. Compared with unlyophilized nanosuspension, the drug loading rate of the PAC lyophilized formulation tended to stabilize during placement, and was 24.6% 6 months later.

PAC prepared by reacting adriamycin (ADM) with pectin is a macromolecular prodrug. Prodrug is an inactive medical precursor, has no or very low activity before being delivered to a target site, is activated under the catalysis of enzyme or non-enzyme action at the target site, and releases active substances, thus exerting pharmacological actions. PAC slowly releases ADM during storage, which has significant impact on in vivo passive targeting anticancer activity. Therefore, compared with the PAC nanosuspension, the nanosuspension lyophilized formulation has obvious advantages.

### 3. CONCLUSION

Taking stability of the drug loading rate of the bulk drugs into consideration, the particle size of nano particles of the nanosuspension is instable during placement, and nano particles tend to further aggregate; the stability of nano particle size of the lyophilized product prepared from the nanosuspension is enhanced; the drug loading rate of the PAC is instable in the nanosuspension with water as a medium, and decreases from 25.8% to 21.6%; and stability of the drug loading rate of the prepared lyophilized product is enhanced. Therefore, the lyophilized product prepared from the PAC nanosuspension can ensure long-term storage stability of the PAC in a better manner.

1. A lyophilized formulation of pectin-adriamycin conjugate prepared by a process comprising preparing a suspension or a nanosuspension containing an insoluble pectin-adriamycin conjugate and a lyophilized support agent, and lyophilizing the suspension or nanosuspension to prepare the lyophilized formulation.

2. The lyophilized formulation of pectin-adriamycin conjugate according to claim 1, wherein the lyophilized support agent is at least one member selected from the group consisting of mannitol, dextran, lactose, sucrose, glucose, sorbitol and sodium chloride.

3. The lyophilized formulation of pectin-adriamycin conjugate according to claim 2, wherein the lyophilized support agent is sucrose.

4. The lyophilized formulation of pectin-adriamycin conjugate according to claim 2, wherein an addition amount of the lyophilized support agent is 40-120 mg/mL.

5. The lyophilized formulation of pectin-adriamycin conjugate according to claim 4, wherein addition amount of the lyophilized support agent is 80 mg/mL.

6. A preparation method for preparing the lyophilized formulation of pectin-adriamycin conjugate according to claim 1, said method comprising:
(a) preparing an insoluble pectin-adriamycin conjugate into a suspension or nanosuspension; and
(b) adding a lyophilized support agent for lyophilization treatment;
(c) separately filling the suspension or nanosuspension in penicillin bottles;
(d) prefreezing at −40°-−80° for 4-8 h;
(e) placing the penicillin bottles in a lyophilizer; and
(f) lyophilizing the suspension or nanosuspension in the penicillin bottles to provide the lyophilized formulation.

7. The preparation method according to claim 6, wherein the prefreezing is conducted at −40°.

8. The preparation method according to claim 6, wherein the prefreezing is conducted for 6 h.

9. The preparation method according to claim 6, wherein the lyophilizing is conducted in the lyophilizer for at least 24 h.

10. The preparation method according to claim 6, wherein the method for preparing the insoluble pectin-adriamycin conjugate into the suspension comprises adding 500-900 mg pectin-adriamycin conjugate and a stabilizer to 100 mL sterile water for injection to obtain a mixture, and grinding the mixture into the suspension.

11. The preparation method according to claim 10, wherein the nanosuspension is prepared after grinding the mixture into a suspension.

12. The preparation method according to claim 11, wherein a high-pressure homogenizer is used for preparing the suspension into the nanosuspension.

13. The preparation method according to claim 10, wherein the stabilizer is at least one member selected from the group consisting of PVP, poloxamer, sodium dodecyl sulfate, polysorbate and hydroxypropyl methyl cellulose.

14. The preparation method according to claim 13, wherein the PVP is PVP K-30.

15. The preparation method according to claim 13, wherein the poloxamer is poloxamer 188 or poloxamer 407.

16. The preparation method according to claim 13, wherein the polysorbate is polysorbate-80.

17. The preparation method according to claim 6, wherein the suspension or nanosuspension preparing step comprises adding 500-900 mg pectin-adriamycin conjugate, 3000-6000 mg PVP K-30 and 500-900 mg poloxamer 188 to 100 mL sterile water for injection to obtain a mixture, and grinding the mixture into the suspension.

18. The preparation method according to claim 17, wherein the suspension or nanosuspension preparing step comprises adding 776 mg pectin-adriamycin conjugate, 4000 mg PVP K-30 and 700 mg poloxamer 188 to 100 mL sterile water for injection to obtain a mixture, and grinding the mixture into the suspension.

19. The preparation method according to claim 17, wherein the lyophilized formulation is prepared by adding 500-900 mg PAC, 3000-6000 mg PVP K-30, 500-900 mg poloxamer 188 and 4000-12000 mg sucrose to 100 mL sterile water for injection before lyophilization.

20. The preparation method according to claim 19, wherein the lyophilized formulation is prepared by adding 776 mg PAC, 4000 mg PVP K-30, 700 mg poloxamer 188 and 8000 mg sucrose to 100 mL sterile water for injection before lyophilization.

21. The preparation method according to claim 6, wherein the suspension or nanosuspension preparing step comprises adding 0.4-1.6 g PVP, 2-6 ml glycerol and 40-60 ml 1-3% lecithin solution as a solvent to 0.3-0.6 g PAC to prepare the suspension.

22. The preparation method according to claim 21, wherein the suspension or nanosuspension preparing step comprises adding 1 g PVP, 3 ml glycerol and 50 ml 2% lecithin solution as a solvent to 0.468 g PAC to prepare the suspension.

23. The preparation method according to claim 6, wherein the suspension or nanosuspension preparing step comprises adding a mixed solvent of 0.4-1.6 g PVP, 40-60 ml water and DMSO to 0.3-0.6 g PAC to prepare the suspension, wherein a water:DMSO ratio equals 0.5-0.85:0.15-0.5.

24. The preparation method according to claim 23, wherein the suspension or nanosuspension preparing step comprises adding a mixed solvent of 1 g PVP, 50 ml water and DMSO to 0.468 g PAC to prepare the suspension.

25. The preparation method according to claim 23, wherein the water:DMSO ratio equals 0.75:0.25.

26. The preparation method according to claim 21, wherein a high-pressure homogenizer is used for treatment of the suspension to prepare the nanosuspension.

27. The preparation method according to claim 26, wherein the high pressure homogenizer is used for treatment to prepare the nanosuspension at a pressure not more than 200 mpa each time.

28. The preparation method according to claim 27, wherein the high pressure homogenizer is used for treatment to prepare the nanosuspension at pressure of 120 mpa for a first time, 180 mpa for a second time and 190 mpa for a third time.

29. The preparation method according to claim 17, wherein the suspension or nanosuspension preparing step comprises:
A preparing PVP K-30 into PVP K-30 solution with sterile water for injection;
B dry grinding and evenly mixing PAC and poloxamer 188, and adding PVP K-30 solution for full grinding;
C adding remaining PVP K-30 solution in several times for grinding;
D performing ultrasonic treatment on the ground suspension for full mixing so as to prepare the suspension; and
E treating the suspension evenly mixed in the step D in a high-pressure homogenizer: controlling pressure of the homogenizer at 4000-8000PSI for cycle treatment of samples for 3-10 min, and then keeping the pressure of the homogenizer at 20000-30000PSI for cycle treatment of the samples for 15-30 min to obtain the nanosuspension.

30. The preparation method according to claim 29, wherein the treatment in the high-pressure homogenizer in the step D comprises controlling the pressure of the homogenizer at 5000PSI for cycle treatment of the samples for 3 min, and then keeping the pressure of the homogenizer at 25000PSI for cycle treatment of the samples for 20 min.
